SEQUENCE LISTING (16 sequences)

SEQ ID NO: 1 - protein, 101 aa, Artificial Sequence
  Monomer of spider silk DP-1A analog protein
GAGRGGQGAGAAAAAAGGAGQGGYGGLGSQGAGRGGLGGQGAGAAAAAAAGGAGQGGLGSQGAGQGAGAAAAAAGGAGQGGYGGLGSQGAGQGGYGGLGSQ

SEQ ID NO: 2 - protein, 101 aa, Artificial Sequence
  Monomer of spider silk DP-1B.9 analog protein
GAGQGGYGGLGSQGAGRGGLGGQGAGAAAAAAAGGAGQGGLGSQGAGQGAGAAAAAAGGAGQGGYGGLGSQGAGRGGQGAGAAAAAAGGAGQGGYGGLGSQ

SEQ ID NO: 3 - protein, 101 aa, Artificial Sequence
  Monomer of spider silk DP-1B.16 analog protein
SQGAGQGGYGGLGSQGAGRGGLGGQGAGAAAAAAAGGAGQGGLGSQGAGQGAGAAAAAAGGAGQGGYGGLGSQGAGRGGQGAGAAAAAAGGAGQGGYGGLG

SEQ ID NO: 4 - protein, 119 aa, Artificial Sequence
  Monomer of spider silk DP-2A analog protein
GPSGPGSAAAAAAGPGQQGPGGYGPGQQGPGGYGPGQQGPSGPGSAAAAAAAAGPGGYGPGQQGPGGYGPGQQGPSGPGSAAAAAAAAAGPGGYGPGQQGPGGYGPGQQGPGGYGPGQQ

SEQ ID NO: 5 - protein, 11 aa, Bacteriophage T7, Gene 10 Protein
MASMTGGQQMG

SEQ ID NO: 6 - protein, 8 aa, Artificial Sequence
  Flag Peptide
DYKDDDDK

SEQ ID NO: 7 - protein, 34 aa, Artificial Sequence
  Consensus repeat sequence representing spider silk analog protein DP-1
  MISC_FEATURE (11)..(11): Xaa = Ser, Gly, or Asn
  MISC_FEATURE (26)..(32): The alanine residues at positions 26-32 may optionally be present or absent.
AGQGGYGGLGXQGAGRGGLGGQGAGAAAAAAAGG

SEQ ID NO: 8 - protein, 21 aa, Artificial Sequence
  A portion of the consensus repeat sequence representing spider silk analog protein DP-1
  MISC_FEATURE (13)..(19): The alanine residues at positions 13 to 19 may optionally be present or absent.
AGRGGLGGQGAGAAAAAAAGG

SEQ ID NO: 9 - protein, 13 aa, Artificial Sequence
  One of the repeat sequences representing spider silk analog protein DP-1
  MISC_FEATURE (11)..(11): Xaa= Ser, Gly, or Asn
AGQGGYGGLGXQG

SEQ ID NO: 10 - protein, 28 aa, Artificial Sequence
  One of the repeat sequences representing spider silk analog protein DP-1
  MISC_FEATURE (8)..(8): Xaa = Ser, Gly, or Asn
  MISC_FEATURE (20)..(26): The alanine residues at positions 20 to 26 may optionally be present or absent.
AGQGGLGXQGAGLGGQGAGAAAAAAAGG

SEQ ID NO: 11 - protein, 47 aa, Artificial Sequence
  Consensus repeat sequence representing spider silk analog protein DP-2
  MISC_FEATURE (44)..(47): The alanine residues at positions 44 to 47 may optionally be present or absent.
GPGGYGPGQQGPGGYGPGQQGPGGYGPGQQGPSGPGSAAAAAAAAAA

SEQ ID NO: 12 - protein, 5 aa, Artificial Sequence
GPGGY

SEQ ID NO: 13 - protein, 5 aa, Artificial Sequence
  Possible deletion in the consensus repeat sequence representing spider silk analog protein DP-2
GPGQQ

SEQ ID NO: 14 - protein, 42 aa, Artificial Sequence
  One of the repeat sequences representing spider silk analog proein DP-2
  MISC_FEATURE (39)..(42): The alanine residues at positions 39 to 42 may optionally be present or absent.
GPGQQGPGGYGPGQQGPGGYGPGQQGPSGPGSAAAAAAAAAA

SEQ ID NO: 15 - protein, 42 aa, Artificial Sequence
  One of the repeat sequences representing spider silk analog protein DP-2
  MISC_FEATURE (39)..(42): The alanine residues at positions 39 to 42 may optionally be present or absent.
GPGGYGPGGYGPGQQGPGGYGPGQQGPSGPGSAAAAAAAAAA

SEQ ID NO: 16 - protein, 37 aa, Artificial Sequence
  One of the repeat sequences representing spider silk analog protein DP-2
  MISC_FEATURE (34)..(37): The alanine residues at positions 34 to 37 may optionally be present or absent.
GPGQQGPGGYGPGGYGPGQQGPSGPGSAAAAAAAAAA

(19) United States
(12) Patent Application Publication
(10) Pub. No.: US 2005/0261479 A1
(43) Pub. Date: Nov. 24, 2005

(54) Method for purifying and recovering silk proteins using magnetic affinity separation

(76) Inventors: Christian Hoffmann, Newark, DE (US); Karsten Keller, Hockessin, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/110,424

(22) Filed: Apr. 20, 2005

Related U.S. Application Data
(60) Provisional application No. 60/566,558, filed on Apr. 29, 2004.

(51) Int. Cl. C07K 14/435; C12P 21/06; C12N 5/06

(52) U.S. Cl. 530/353; 435/348; 435/69.1

(57) ABSTRACT

A method for the purification of recombinant silk proteins from a sample using magnetic affinity separation is described. The recombinant silk protein is expressed with an affinity tag which has a high binding affinity for an affinity ligand immobilized on magnetic particles. In the method, the processes of clarification of the crude silk protein extract, concentration of the product and purification of the product are combined in a single step involving the affinity capture of the spider silk protein onto the magnetic particles directly from the extract. The product yields are improved due to the reduced number of steps in the purification process.

This application claims the benefit of U.S. Provisional Patent Application 60/566558, filed Apr. 29, 2004.

This invention was made with Government support under contract number DE-FC36-99G010287 awarded by DOE. The Government has certain rights in this invention.

### FIELD OF THE INVENTION

The invention relates to the field of purification and recovery of proteins from a sample. More specifically, the invention relates to a method for the purification and recovery of recombinant silk protein using magnetic affinity adsorbent particles and magnetic separation.

### BACKGROUND OF THE INVENTION

Silks are some of the strongest natural fibers known, rivaling high performance synthetic fibers in mechanical properties. Strong natural fibers with high tensile strength and elasticity are useful for many applications, including high strength fibers for textile applications and composite materials, such as parachutes, sails and body armor. Additionally, silk proteins have low immunogenic and allergenic potential, making them suitable for medical applications such as wound sutures, membranes, surfaces for cultivated cells, and as a scaffold for artificial organs. Silk proteins self-assemble in solution (Winkler et al., Int. J. Biol. Macromol. 24:265-270 (1999)), making them useful in applications requiring film formation and surface coating, such as skin and hair care products, particle coating, and in wound dressings (see for example, Fahnestock et al. copending, commonly owned. U.S. patent application Ser. No. 10/772124, U.S. Patent Application Publication No. 2004/0170590).

Silks are produced by over 30,000 species of spiders and by many insects particularly in the order Lepidoptera (Foelix, Biology of Spiders, Harvard University Press Cambridge, Mass. (1992)). Few of these silks have been studied in detail. The cocoon silk of the domesticated silkworm Bombyx mori and the dragline silk of the orb-weaving spider Nephila clavipes are among the best characterized. Silk production from silkworms and from cultivated spiders is labor intensive and time consuming and therefore prohibitively expensive. Consequently, recombinant DNA technology has been used to produce silk proteins. Ohshima et al. (Proc. Natl. Acad. Sci. USA, 74:5363-5367 (1977)) report the cloning of the silk fibroin gene complete with flanking sequences of the silkworm Bombyx mori into E. coli. Petty-Saphon et al. (EP 0230702) disclose the recombinant production of silk fibroin and silk sericin from a variety of hosts including E. coli, Saccharomyces cerevisiae, Pseudomonas sp., Rhodopseudomonas sp., Bacillus sp., and Strepomyces sp.

The production of recombinant spider silk proteins is also known. Xu et al. (Proc. Natl. Acad. Sci. U.S.A., 87:7120-7124 (1990)) report the determination of the sequence for a portion of the repetitive sequence of a dragline spider silk protein, Spidroin 1, from the spider Nephila clavipes, based on a partial cDNA clone. Hinman and Lewis (J. Biol. Chem. 267:19320-19324 (1992)) report the sequence of a partial cDNA clone encoding a portion of the repeating sequence of a second fibroin protein, Spidroin 2, from dragline silk of Nephila clavipes. Lewis et al. (U.S. Pat. Nos. 5,728,810 and 5,989,894) disclose the expression of spider silk proteins including protein fragments and variants of Nephila clavipes from transformed E. coli. Two distinct proteins were independently identified and cloned and were distinguished as silk protein 1 (Spidroin 1) and silk protein 2 (Spidroin 2). cDNA clones encoding minor ampullate spider silk proteins and the expression thereof are described by Lewis et al. (U.S. Pat. Nos. 5,733,771 and 5,756,677). Lewis et al. (U.S. Pat. No. 5,994,099) describe the cloning of cDNA encoding the flagelliform silk protein from an orb-web spinning spider. Lewis et al. (WO 03/020916) describe the cloning of spider silk proteins from various other spiders. Fahnestock (U.S. Pat. No. 6,268,169) describes novel spider silk analog proteins derived from the amino acid consensus sequence of repeating units found in the natural spider dragline of Nephila clavipes. The synthetic spider dragline was produced from E. coli, Bacillus subtilis, and Pichia pastoris recombinant expression systems. Spider silk proteins and analog proteins have also been expressed in plants (Yang, WO 01/90389, and U.S. Pat. No. 6,608,242, and Scheller et al., DE 10113781). Additionally, spider silk proteins have been expressed in mammalian cells (Lazaris et al., Science 295:472476 (2002)) and in transgenic animals (Clark et al. in U.S. Patent Application Publication No. 2001/0042255 and Karatzas et al. WO 99/47661).

Methods for the recovery and purification of recombinant silk proteins are known in the art. These methods involve multistep processes that result in significant product losses. Moreover, the purification of recombinant spider silk proteins from microbial sources is complicated by precipitation of the proteins due to the self-assembly into insoluble microfibrils (Winkler et al. Int. J. Biol. Macromol. 24:265-270 (1999), and Arcidiacono et al. Macromolecules 35:1262-1266 (2002)). Therefore, the product yields obtained with these methods are too low for economical large-scale commercial production of spider silk proteins. For example, the use of affinity chromatography for the purification of recombinant spider silk proteins containing a histidine tag using a Nickel affinity resin is described by Fahnestock (U.S. Pat. No. 6,268,169) and Lewis et al. (Protein Expression and Purification 7:400-406 (1996)). In that method, the cell extract must be clarified by centrifugation or filtration prior to application to the chromatography column.

Mello et al. (WO 01/53333) describe a method for purifying spider silks and other structural proteins which involves lysing the host cells in the presence of an organic acid. After cell lysis, a number of centrifugation and filtration steps are required prior to purification by anion exchange or affinity chromatography.

Karatzas et al. (WO 03/057720) describe various methods for the recovery and purification of biofilament proteins, i.e., silk proteins, from biological fluids. The methods described utilize a clarification step, such as tangential flow filtration or centrifugation, after cell lysis. Then, the proteins are purified by precipitation, or chromatographic methods such as anion exchange, cation exchange, size exclusion, affinity, and hydrophobic interaction chromatography.

Fahnestock et al. in copending, commonly owned U.S. patent application Ser. No. 10/704337, U.S. Patent Application Publication No. 2004/0132978, describe a method for recovering and purifying spider silk proteins in soluble form using precipitation at low temperature. Although this method results in a soluble spider silk protein precipitate that can be redissolved in water without the use of harsh denaturing agents, higher product yields are still required for economical commercial production.

All of the above methods require clarification of the cell extract before recovery of the spider silk protein. This clarification step complicates the purification process and results in low product yields, typically 35% or less.

The use of magnetic affinity separation for the purification of recombinant proteins is known in the art (e.g., O'Brien et al. J. Biotechnol. 54:53-67 (1997), Frenzel et al. J. Chromatgr. B 793:325-329 (2003), and Nishiya et al. Protein Expression and Purification 25:426-429 (2002)). However, the use of magnetic affinity separation for the purification of silk proteins has not been described.

The problem to be solved, therefore, is the need for a purification method for recombinant silk proteins that results in increased product yield, so that commercial production is more economical. Applicants have solved the stated problem through the discovery of a magnetic affinity separation method for recovering and purifying recombinant silk proteins that results in product yields of up to 71%.

### SUMMARY OF THE INVENTION

The invention provides a method for the purification of silk protein from a sample comprising:
a) providing a sample comprising at least one silk protein having an affinity tag in the presence of contaminating proteins;
b) contacting the sample with magnetic particles comprising an affinity ligand having a binding affinity for the affinity tag for a time sufficient for capture of the at least one silk protein onto the magnetic particles;
c) separating the magnetic particles from the sample by applying a magnetic field; and
d) recovering the silk protein from the magnetic particles by contacting the particles with an elution solution.

In another embodiment, the invention provides a method for the purification of silk protein from a host cell comprising:
a) providing a host cell comprising at least one silk protein having an affinity tag;
b) disrupting the host cell to release the at least one silk protein and produce a crude silk protein extract;
c) contacting the crude silk protein extract with magnetic particles comprising an affinity ligand having a binding affinity for the affinity tag for a time sufficient for capture of the silk protein onto the magnetic particles;
d) separating the magnetic particles from the crude silk protein extract by applying a magnetic field; and
e) recovering the silk protein from the magnetic particles by contacting the particles with an elution solution.

### BRIEF DESCRIPTION OF THE FIGURES AND SEQUENCE DESCRIPTIONS

The invention can be more fully understood from the following detailed description, figures, and the accompanying sequence descriptions, which form a part of this application.

FIG. 1 is the gel image of the electropherogram obtained for various samples during the purification of DP-2A spider silk analog protein from a crude silk protein extract using polyglutaraldehyde-coated ferrite magnetic particles, as described in Example 1.

FIG. 2 is the gel image of the electropherogram obtained for various samples during the purification of DP-2A spider silk analog protein from a crude silk protein extract using poly(vinyl alcohol)-coated magnetic particles, as described in Example 2.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NO:1 is the amino acid sequence of the monomer of the spider silk DP-1A analog protein.

SEQ ID NO:2 is the amino acid sequence of the monomer of the spider silk DP-1B.9 analog protein.

SEQ ID NO:3 is the amino acid sequence of the monomer of the spider silk DP-1B.16 analog protein.

SEQ ID NO:4 is the amino acid sequence of the monomer of the spider silk DP-2A analog protein.

SEQ ID NO:5 is the amino acid sequence of the T7 Tag®.

SEQ ID NO:6 is the amino acid sequence of the Flag® peptide.

SEQ ID NO:7 is the amino acid sequence of the consensus repeat sequence representing spider silk analog protein DP-1.

SEQ ID NO:8 is the amino acid sequence of a portion of the consensus repeat sequence representing spider silk analog protein DP-1.

SEQ ID NO:9 is the amino acid sequence of one of the repeat sequences representing the spider silk analog protein DP-1.

SEQ ID NO:10 is the amino acid sequence of one of the repeat sequences representing the spider silk analog protein DP-1.

SEQ ID NO:11 is the amino acid sequence of the consensus repeat sequence representing spider silk analog protein DP-2.

SEQ ID NOs:12 and 13 are the amino acid sequences of possible deletions in the consensus repeat sequence representing spider silk analog protein DP-2.

SEQ ID NOs:14-16 are the amino acid sequences of three of the repeat sequences representing the spider silk analog protein DP-2.

### DETAILED DESCRIPTION OF THE INVENTION

The invention provides a method for the recovery and purification of recombinant silk proteins using magnetic affinity separation. In the method, the processes of clarification of the crude silk protein extract, concentration of the product and purification of the product are combined in a single step involving the affinity capture of the silk protein onto magnetic particles, directly from the extract. The product yields are improved due to the reduced number of steps in the purification process, compared to other purification techniques.

The invention is useful because silk proteins have utility in producing high strength fibers for textile applications and composite materials, such as parachutes, sails and body armor; in medical applications, such as wound sutures, wound dressings, membranes, surfaces for cultivated cells, and as a scaffold for artificial organs; and as film-forming agents in personal care products, such as skin and hair care products.

The following definitions are used herein and should be referred to for interpretation of the claims and the specification.

“Nucleic acid” refers to a molecule which can be single stranded or double stranded, composed of monomers (nucleotides) containing a sugar, phosphate and either a purine or pyrimidine. In bacteria, lower eukaryotes, and in higher animals and plants, “deoxyribonucleic acid” (DNA) refers to the genetic material while “ribonucleic acid” (RNA) is involved in the translation of the information from DNA into proteins.

The terms “polypeptide” and “protein” are used interchangeably.

The term “peptide” is used to describe a subunit of a polypeptide or protein formed by hydrolysis.

“Gene” refers to a nucleic acid fragment that effects the production of a specific protein, including regulatory sequences preceding (5″ non-coding sequences) and following (3″ non-coding sequences) the coding sequence.

“Codon” refers to a unit of three nucleotides that encodes a single amino acid.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” host cells.

The term “expression” as used herein is intended to mean the transcription and translation to gene product from a gene coding for the sequence of the gene product. In the expression, a DNA chain coding for the sequence of gene product is first transcribed to a complementary RNA which is often a messenger RNA and, then, the thus transcribed messenger RNA is translated into the above-mentioned gene product if the gene product is a protein.

The terms “silk variant protein” and “silk analog protein” are used interchangeably herein to refer to a recombinant protein, the amino acid sequence of which is based on repetitive sequence motifs and variations thereof that are found in a known natural silk protein.

The term “full length variant protein” refers to any silk variant protein encoded by a synthetic gene, which has been constructed by the assembly and polymerization of a DNA monomer.

The term “DNA monomer” refers to a DNA fragment consisting of between 300 and 400 bp which encodes one or more repeating amino acid sequences of a silk variant protein.

The term “peptide monomer”, or “polypeptide monomer” refers to the amino acid sequence encoded by a DNA monomer.

The term “DP-1 analog” refers to any spider silk variant derived from the amino acid sequence of the natural Protein 1 (Spidroin 1) of Nephila clavipes.

The term “DP-2 analog” refers to any spider silk variant derived from the amino acid sequence of the natural Protein 2 (Spidroin 2) of Nephila clavipes.

As used herein the following abbreviations are used to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Asparagine or aspartic acid | Asx | B |
| Cysteine | Cys | C |
| Glutamine | Gln | Q |
| Glutamine acid | Glu | E |
| Glutamine or glutamic acid | Glx | Z |
| Glycine | Gly | G |
| Histidine | His | H |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The term “affinity tag” refers to a peptide or polypeptide that is incorporated into a fusion protein to facilitate purification of the protein.

The term “affinity ligand” refers to a moiety that has high binding affinity and selectivity to the affinity tag. In the present invention, the affinity ligand is immobilized on magnetic particles.

The terms “histidine tag” and “His tag” are used interchangeably herein to refer to an affinity tag consisting of a polyhistidine sequence comprising two or more histidine residues.

The terms “growth medium” and “fermentation medium” are herein used interchangeably to refer to an aqueous solution containing nutrients for culturing microorganisms. The growth medium may additionally contain the microorganism, the product produced by the microorganism, metabolic intermediates, and other components such as salts, vitamins, amino acids, cofactors, and antibiotics.

All ranges given herein include the end of the ranges and also all the intermediate range points.

The present invention provides several embodiments for the purification and recovery of silk proteins from a sample containing other contaminating proteins using magnetic affinity separation.

Silk Proteins

A silk protein is herein defined as a recombinant silk protein or analog, having a glycine-rich sequence, herein referred to as the soft segment, alternating with an oligomer of polyalanine, herein referred to as the hard segment, in which at least about 20% of the soft segment is composed of glycine and about 70-100% of the hard segment is composed of alanine. The length of the hard segment is between about 5 to 100 amino acids, while the length of the soft segment is between about 5 to 300 amino acids.

The recombinant silk proteins include, but are not limited to, spider silk proteins and spider silk analog proteins. Analog silk proteins are herein defined as polypeptides that imitate the repeating units of amino acids of natural silk proteins. The terms “analog silk protein” and “silk variant protein” are herein used interchangeably. For example, the silk protein may be recombinant dragline spider silk protein, specifically, Spidroin 1 or Spidroin 2, or variants thereof, originating from the major ampullate gland of Nephila clavipes, as described by Lewis et al. in U.S. Pat. Nos. 5,728,810 and 5,989,894, incorporated herein by reference. Additionally, the silk protein may be the recombinant spider silk proteins originating from the minor ampullate gland of Nephila clavipes, or variants thereof, as described by Lewis in U.S. Pat. Nos. 5,733,771 and 5,756,677, incorporated herein by reference. The silk protein may also be the recombinant silk protein originating from the flagelliform gland of Nephila clavipes, or variants thereof, as described by Lewis in U.S. Pat. No. 5,994,099, incorporated herein by reference. Additionally, the silk protein may be recombinant spider silk protein originating from the major ampullate glands of Nephila madagascariensis, Nephila senegalensis, Tetragnatha kauaiensis, Tetragnatha versicolor, Argiope aurantia, Argiope trifasciata, Gasteracantha mammosa, and Latrodectus geometricus, the flagelliform glands of Argiope trifasciata, the ampullate glands of Dolomedes tenebrosus, the silk glands of Plectreurys tristis and the mygalomorph Euagrus chisoseus, as described by Lewis et. al. in WO 03/020916, incorporated herein by reference. The silk protein may also be one or more of those described by Lewis et al. in Protein Expression and Purification 7:400-406 (1996), or by Prince et al., in Biochemistry 34:10879-10885 (1995), or by Winkler et al. in Int. J. Biol. Macromol. 24:265-270 (1999). Moreover, the silk protein may be a variant designed to possess certain beneficial properties. For example, the silk protein variant may be designed to have increased elasticity by elongating the glycine-rich (soft) segment, as described in patent applications WO 9116351 and EP 452,925, both of which are incorporated herein by reference. Conversely, it is possible to reduce the elasticity of the silk protein by shortening the glycine-rich segment. Similarly, the replacement of the glycine residues with serine residues results in a less rigid silk protein.

In one embodiment, the silk proteins are spider silk analog proteins, as described by Fahnestock in U.S. Pat. No. 6,268,169, incorporated herein by reference. That disclosure describes analog proteins of the natural dragline spider silk Spidroin 1 (DP-1) and Spidroin 2 (DP-2) proteins of Nephila calvipes. Two analogs of DP-1 were designed and designated DP-1A and DP-1B. DP-1A is composed of a tandemly repeated 101-amino acid sequence. The 101-amino acid “peptide monomer”, given as SEQ ID NO:1, comprises four repeats which have different patterns that reflect the variation of the individual repeating units of DP-1 from the consensus sequence. This 101-amino acid long peptide monomer (SEQ ID NO:1) was repeated from 1 to 16 times in a series of analog proteins. DP-1B was designed by reordering the four repeats within the monomer of DP-1A. Two sets of genes using different codons were designed to produce DP-1B ,specifically DP-1B.9 and DP-1B.16. The resulting amino acid monomer sequences are given as SEQ ID NO:2 for DP-1B.9 and SEQ ID NO:3 for DP-1B.16. The sequence of DP-1B matches the natural sequence of Spidroin 1 more closely over a more extended segment than does DP-1A. The DP-1 amino acid sequences and similar analogs may be represented by the following consensus repeat formula (SEQ ID NO:7):

| | |
|---|---|
| [AGQGGYGGLGXQGAGRGGLGGQGAGAnGG]z | (1) |

wherein X=S, G or N; n=0-7 and z=1-75, and wherein the value of z determines the number of repeats in the variant protein. The formula encompasses variations selected from the group consisting of: (a) when n=0, the sequence encompassing AGRGGLGGQGAGAnGG, (given as SEQ ID NO:8) is deleted giving the sequence as set forth in SEQ ID NO:9; (b) deletions other than the poly-alanine sequence, limited by the value of n will encompass integral multiples of three consecutive residues; (c) the deletion of GYG in any repeat is accompanied by deletion of GRG in the same repeat, resulting in the sequence given as SEQ ID NO:10; and (d) where a first repeat where n=0 is deleted, the first repeat is preceded by a second repeat where n=6; and wherein the full-length protein is encoded by a gene or genes and wherein said gene or genes are not endogenous to the Nephila clavipes genome.

Synthetic analogs of DP-2 were designed to mimic both the repeating consensus sequence of the natural protein and the pattern of variation among individual repeats of Spidroin 2. The analog DP-2A, given as SEQ ID NO:4, is composed of a tandemly repeated 119-amino acid sequence. This 119-amino acid “peptide monomer” comprises three repeats which have different patterns. This 119-amino acid long peptide monomer was repeated from 1 to 16 times in a series of analog proteins. The DP-2 amino acid sequence and similar analogs may be represented by the following consensus repeat formula (SEQ ID NO:11):

| | |
|---|---|
| [GPGGYGPGQQGPGGYGPGQQGPGGYGPGQQGPSGPGSAn]z | (2) |

wherein n=6-10 and z=1-75 and wherein, excluding the poly-alanine sequence, individual repeats differ from the consensus repeat sequence by deletions of integral multiples of five consecutive residues consisting of one or both of the pentapeptide sequences GPGGY (SEQ ID NO:12) or GPGQQ (SEQ ID NO:13), resulting in the sequences given as SEQ ID NOs:14-16, and wherein the full-length protein is encoded by a gene or genes and wherein the gene or genes are not endogenous to the Nephila clavipes genome.

The silk proteins may be prepared by transformed prokaryotic or eukaryotic host cells, including bacterial, yeast, fungi, algae, plant, and mammalian hosts, using standard recombinant DNA techniques. These recombinant DNA techniques are described by Sambrook, J., Fritsch, E. F. and Maniatis, T. Molecular Cloning: A Laboratory Manual, Second Edition, Cold Spring Harbor Laboratory Press, Cold Spring Harbor (1989), and Clark et al., Plant Molecular Biology, A Laboratory Manual, Springer-Verlag, Berlin, Heidelberg (1997), both of which are incorporated herein by reference.

In one embodiment, the silk proteins are expressed in plants such as Arabidopsis or soy somatic embryos as described by Yang in U.S. Pat. No. 6,608,242, incorporated herein by reference. Additionally, silk analog proteins may be expressed in the endoplasmic reticulum of the leaves or tubers of transgenic tobacco and potato plants, as described in patent application DE 10113781, incorporated herein by reference. Other suitable plant hosts include, but are not limited to, soybean, rapeseed (Brassica napus, B. campestris), pepper, sunflower (Helianthus annus), cotton (Gossypium hirsutum), corn, alfalfa (Medicago sativa), wheat (Triticum sp), barley (Hordeum vulgare), oats (Avena sativa, L), sorghum (Sorghum bicolor), rice (Oryza sativa), cruciferous vegetables (broccoli, cauliflower, cabbage, parsnips, etc.), melons, carrots, celery, parsley, tomatoes, strawberries, peanuts, grapes, grass seed crops, sugar beets, sugar cane, beans, peas, rye, flax, hardwood trees, softwood trees, and forage grasses.

In another embodiment, the silk proteins are expressed in mammalian cells such as bovine mammary epithelial alveolar cells or baby hamster kidney cells, as described by Lazaris et al. in Science 295:472-476 (2002), incorporated herein by reference. In both mammalian expression systems, the silk proteins were excreted into the culture media. In another embodiment, the silk proteins are expressed in transgenic animals, which secrete the proteins in their milk or urine, as described by Clark et al. in published U.S. Patent Application Publication No. 2001/0042255 and Karatzas et al. in patent application WO 99/47661, both of which are incorporated herein by reference.

In another embodiment, the silk proteins are expressed in microbial systems. Suitable microbial expression systems include, but are not limited to, Escherichia, Bacillus, Saccharomyces, Schizosaccharomyces, Pichia, Aspergillus, and Streptomyces. In another embodiment, the silk analog proteins DP-1A, DP-1B, and DP-2A are expressed in E. coli, Bacillus subtilis, or Pichia pastoris, as described by Fahnestock (U.S. Pat. No. 6,268,169). In another embodiment, the spider silk analog protein DP-2A is expressed in E. coli.

Affinity Tags

The use of affinity tags for the purification of proteins using affinity chromatography is well known in the art (see for example, Sassenfield, Trends Biotechnol. 8:88-93 (1990), Sherwood, Trends Biotechnol. 9:1-3 (1991), Constans, The Scientist 16(4):37 (2002), and Nilsson et al., Protein Expression and Purification 11:11-16 (1997)). The affinity tag is incorporated into the fusion protein to provide a binding site having a high affinity for a counter-affinity ligand, which is immobilized on a solid support. Any affinity tag that provides a high selectivity and high affinity towards a counter-ligand, which can be immobilized on the surface of magnetic particles, is suitable for use in the instant invention. Examples of suitable affinity tags include, but are not limited to an antibody; an antibody fragment; glutathione S-transferase (GST tag); a polyhistidine sequence comprising two or more histidine residues (His tag); streptavidin; avidin; Protein A; Protein G; maltose-binding protein; the T7 Tag® (available from Novagen, Inc. (Madison, Wis.), given as SEQ ID NO:5; the Flag® peptide sequence (available from Sigma Chemical Co., St Louis, Mo.), given by SEQ ID NO:6; and mixtures thereof.

The affinity tag is incorporated into the recombinant silk protein using standard recombinant DNA techniques, as described by Sambrook, J., Fritsch, E. F. and Maniatis, T., supra. Specifically, DNA encoding the polypeptide affinity tag is added to either the 5′ or 3′ end of the gene encoding the spider silk protein. Additionally, vectors that encode affinity tags for use in the production of affinity-tagged recombinant proteins are available commercially from a number of sources, including, Clontech Laboratories, Palo Alto, Calif. (His tag), Amersham Biosciences, Piscataway, N.J. (GST tag), Invitrogen, Carlsbad, Calif. (His tag and GST tag), Qiagen, Valencia, Calif. (His tag), Sigma, St. Louis, Mo. (Flag® peptide), Novagen Inc, Madison, Wis. (GST tag, His tag, T7 Tag®), and New England Biolabs, Beverly, Mass. (maltose-binding binding protein tag). Optionally, an enzymatic or chemical cleavage site may be included in the silk fusion protein to permit removal of the affinity tag after the protein is recovered.

Magnetic Particles

The magnetic particles for use in the instant invention contain at least two components, a base magnetic particle and an affinity ligand that is immobilized on the particle surface. Any suitable magnetic base particle that allows the immobilization of the affinity ligand may be used. The base particle may comprise a magnetic material, or a mixture of a magnetic material and a non-magnetic material. As used herein, the term “magnetic” refers to ferromagnetic, ferrimagnetic, or superparamagnetic material properties. The term “non-magnetic” refers to diamagnetic or paramagnetic material properties. The magnetic material may be a metal, an alloy, a mineral, or a mixture thereof. Examples of suitable magnetic materials include, but are not limited to magnetite (Fe₃O₄), maghemite (Fe₂O₃), FePt, SrFe, iron, cobalt, nickel, chromium dioxide, ferrites, or mixtures thereof. The non-magnetic material may be a polymer, metal, glass, alloy, mineral, or mixture thereof. The non-magnetic material may be a coating around the magnetic material or the magnetic material may be distributed in the continuous phase of the non-magnetic material. Suitable non-magnetic materials for coating magnetic materials or for entrapping magnetic materials include, but are not limited to polymers such as polyethylene glycol, polymethacrylate, polymethylmethacrylate, polystyrene, polyethylenimine, polyglutaraldehyde, polyvinyl alcohol, polyvinyl acetate; and gels such as polyacrylamide, agarose, chitosan, and alginate. Typically, if a combination of a magnetic material and a non-magnetic material is used, the magnetic material is at least about 50% by weight of the total composition, in addition at least about 70% by weight of the total composition, and further in addition at least about 80% by weight of the total composition.

Ideally, for use in the instant invention, the magnetic particle should provide the following features: high saturation magnetization and low or vanishing remnant magnetization, non-porous particle structure, chemically stability in all process and cleaning conditions, high surface area per particle volume, a hydrophilic particle surface to minimize nonspecific binding of proteins, and a high density of surface chemical groups per unit surface area. Suitable magnetic particles are available commercially from a number of sources, including Advanced Magnetics (Cambridge, Mass.), Chemicell (Berlin, Germany), Qiagen (Valencia, Calif.), Perseptive Diagnostics (Cambridge, Mass.), Chemagen Biopolymer Technologie AG (Baesweiler, Germany), and Miltenyi Biotech (Auburn, Calif.). Additionally, magnetic particles may be prepared using methods know in the art (see for example Moffat et al. in Minerals Engineering 7:1039-1056, (1994) and references therein, Hubbuch et al. Biotechnol. Bioeng. 79:301-313 (2002), Furukawa et al. U.S. Patent Application Publication No. 2003/0175826, and Bucak et al. Biotechnol. Prog. 19:477-484 (2003)).

In one embodiment, the magnetic particles are ferrite particles coated with polyglutaraldehyde, prepared as described by Hubbuch et al., supra.

In another embodiment, the magnetic particles are poly(vinyl alcohol)-coated particles, M-PVA 01x, obtained from Chemagen.

The magnetic particles have an average size ranging from about 2 nanometers to about 50 micrometers in diameter, in addition ranging from about 100 nanometers to about 10 micrometers in diameter, further in addition ranging from about 500 nanometers to about 5 micrometers.

The specific affinity ligand immobilized on the magnetic particles depends on the affinity tag incorporated into the silk protein. The affinity ligand must have a high affinity and specificity for the affinity tag used. Suitable affinity ligands for various affinity tags are well known in the art (see for example, Sassenfield, supra, Sherwood, supra, Constans, supra, and Nilsson et al., supra). For example, when an antibody or antibody fragment is used as the affinity tag, an IgG binding protein, such as Protein A or Protein G, or an antigen to the antibody may be used as the affinity ligand. Conversely, when Protein A or Protein G is used as the affinity tag, an IgG or IgG fragment is used as the affinity ligand. Other affinity ligands include but are not limited to glutathione, used with a GST tag; biotin, used with a streptavidin or avidin tag; anti-Flag® antibody, used with the Flag® peptide tag; amylose, used with a maltose-binding protein tag; anti-T7 Tag® antibody, used with T7 Tag®; and metal chelates or anti-His tag antibody, used with His tags. Metal chelates that may be used as affinity ligands with His tags include, but are not limited to chelates of nitrilotriacetic acid (NTA) or iminodiacetic acid (IDA) with heavy metal bivalent ions such as Cu(II), Ni(II), Zn(II), Co(II), Hg(II), or Fe(II). Additionally, mixtures of the aforementioned affinity tags may be used.

In one embodiment, a His tag is used as the affinity tag with an IDA-Cu chelate as the affinity ligand. In another embodiment, a His tag is used as the affinity tag with a NTA-Ni chelate as the affinity ligand.

The affinity ligand may be immobilized onto the magnetic particles using any method known in the art (see for example Hermanson, Immobilized Affinity Ligand Techniques, Academic Press, San Diego, 1992, Scouten, Affinity Chromatography, John Wiley and Sons, New York, 1981, Chapter 3, and Mohr et al., Affinity Chromatography, Practical and Theoretical Aspects, Marcel Dekker, New York, 1985, Chapter 4). For example, the affinity ligand may be immobilized by adsorption onto uncoated magnetic particles or polymer-coated magnetic particles. Alternatively, the affinity ligand may be covalently attached to the magnetic particles using any linking chemistry known in the art. The linking chemistry used depends on the functional groups present on the magnetic particles or on the polymer coating on the magnetic particles and the ligand to be immobilized. Amine and carboxylic acid groups on the protein may be used to covalently attach the protein to the magnetic particles. For example, proteins may be immobilized onto ferric oxide particles using silane chemistry, as described by Kausch et al. WO 92/08133. Additionally, amine, carboxylic acid, aldehyde, and hydroxyl groups on polymer-coated magnetic particles may be used to covalently attach proteins and other affinity ligands using well known methods such as carbodiimide coupling, esterification reactions involving hydroxyl groups and carboxylic acid groups, diisocyanate coupling through primary amine groups and hydroxyl groups, and the use of bifunctional reagents having reactive groups such as amine, aldehyde, epoxide, isocyanate, or N-hydroxysuccinimide ester. Metal ion chelates may be immobilized onto magnetic particles using the methods described by O'Brien et al. (J. Biotechnol. 50:13-25 (1996)). In addition, magnetic particles with immobilized affinity ligands are commercially available from sources such as Chemicell (SiMAC beads having immobilized Ni ions), Qiagen (magnetic agarose beads with immobilized NTA-Ni), Advanced Magnetics (magnetic particles with immobilized antibodies and biotinylated magnetic particles) and Miltenyi (immobilized anti-His tag antibody). Amine-terminated iron oxide particles, which facilitate attachment of affinity ligands, are also available commercially from Perseptive Diagnostics.

In one embodiment, IDA is immobilized onto polyglutaraldehyde-coated ferrite magnetic particles and charged with copper (II) ions.

In another embodiment, IDA is immobilized onto PVA-coated magnetic particles and charged with copper (II) ions.

Purification of Silk Protein

The sample for use in the method of the present invention may be any aqueous solution comprising at least one silk protein, having an affinity tag, in the presence of other contaminating proteins. For example the sample may be a crude silk protein extract, a fermentation medium, a culture medium, or a biological fluid such as milk or urine. The procedure used to purify the silk protein will vary somewhat depending on its source, as described below.

Microbial Production of Silk Protein

In one embodiment, transformed microbial cells, engineered to produce silk proteins or variants thereof, are grown in a suitable growth medium to high density. The growth medium used is not critical. Any conventional medium may be used, including but not limited to, LB medium (containing tryptone, yeast extract, and NaCl), complex media containing organic nitrogen sources such as yeast extract, or minimal or defined media. When the cell growth has reached the desired level, the cells are harvested by means, including but not limited to, centrifugation or filtration. In one embodiment, the cells are harvested by centrifugation, typically at 2500 to 25,000×g. The cells may be frozen at about −20° C. before proceeding.

The cell paste is suspended in an appropriate buffered aqueous solution, such as phosphate or Tris buffer, having a pH that does not denature the silk protein. Typically, a pH of about 7.5 is used. The cells may be disrupted by any convenient means including mechanical, chemical or enzymatic methods to give a crude silk protein extract. Mechanical methods include, but are not limited to, sonication, homogenation, irradiation, pressing, freeze-thawing or grinding. Chemical methods include, but are not limited to, treatment with alkali such as sodium hydroxide, treatment with detergents such as sodium dodecyl sulfate (SDS) or suspending the cells in a hypotonic solution to induce lysis via osmotic shock. The chemicals used should be compatible with the subsequent affinity purification step and the affinity ligand used on the magnetic particles. In the case of metal chelate ligands, a chelating agent, such as ethylenediaminetetraacetic acid (EDTA), should not be used for cell disruption because the chelating agent will remove the metal ion from the metal chelate ligand. Enzymatic methods include treatment with a lytic enzyme such as lysozyme, although other lytic enzymes are also effective.

In one embodiment, lysozyme is added to disrupt the cells, followed by the addition of DNase I to digest DNA. This mixture is incubated at about 4° C. to about 37° C. for between 15 to 60 min.

In another embodiment, a combination of treatment with lysozyme and sonication is used. Lysozyme is added to disrupt the cells, followed by the addition of DNase I to digest DNA. This mixture is incubated at about 4° C. to about 37° C. for about 30 min. The cells are then sonicated on ice.

During and subsequent to the lysis step, care should be taken to minimize mechanical stress on the crude silk protein extract because silk proteins are known to be shear-sensitive. For example, aggressive mixing of the silk protein extract may lead to protein precipitation, resulting in lower yields. In the presence of the magnetic particles, aggressive mixing may lead to co-precipitation of the particles, resulting in lower yields and lower product purity.

The magnetic particles with immobilized affinity ligand are added directly to the crude silk protein extract to capture the silk protein. In contrast to other purification methods, there is no need to remove the cell debris. Optionally, salt may be added to the extract to give a salt concentration of about 0.02 to about 1 mole per liter in order to increase the ionic strength. The high ionic strength suppresses nonspecific binding of proteins to the magnetic particles due to ionic interactions. However, if the ionic strength is too high, typically above 1 M, precipitation of the silk protein or nonspecific hydrophobic binding of proteins to the magnetic particles may occur, thereby decreasing product yield and purity. Additionally, agents with high affinity to the magnetic particle surface that would compete with the binding of the silk protein, should not be added to the extract. Specifically, agents that cause an alteration of the affinity ligand, causing a decrease in its binding affinity or selectivity towards the silk protein, should not be present in the extract.

The amount of magnetic particles added to the crude silk protein extract depends on the concentration of the silk protein and on the binding capacity of the magnetic particles. The amount of magnetic particles used should be approximately equal to or slightly greater than the amount of the particles required to completely bind all of the silk protein in the extract. Typically, an excess of 5-10% of the magnetic particles is used. The amount of magnetic particles needed to completely bind all of the silk protein for any particular purification may be estimated by dividing the total amount of silk protein in the extract by the binding capacity of the magnetic particles. The amount of silk protein in the extract may be estimated using any suitable protein assay. The binding capacity of the magnetic particles may be determined empirically by exposing known amounts of the magnetic particles to varying amounts of silk protein or by exposing known amounts of silk protein to varying amounts of magnetic particles. If much lower amounts of the magnetic particles are used, the capture of the silk protein will be incomplete, resulting in low product yield. The use of much higher amounts of magnetic particles is inefficient and may lead to increased nonspecific binding of unwanted proteins, resulting in decreased purity of the silk protein.

The magnetic particles are mixed with the crude silk protein extract for a time sufficient to capture the silk protein. The time required depends on many factors, including the concentration of silk protein in the extract, the volume of extract, the amount of magnetic particles, the size of the magnetic particles, and the manner of mixing used. For example, when using non-porous, micrometer-sized particles, the binding rate is limited by external mass transfer of the silk protein to the particle surface. Typically, the time required to efficiently capture the silk protein using approximately 1 micrometer size, non-porous particles may be expected to be about 2 to 5 minutes. For porous particles, internal mass transfer may be limiting. Therefore, for approximately 100-micrometer size, porous particles, a few hours may be required to capture the silk protein. The time required for any purification may be determined empirically, for example, by monitoring the protein concentration in the extract as a function of time.

The extract/magnetic particle suspension may be mixed in any suitable reaction vessel. For example, a stirred tank reactor or a fluidized bed reactor, such as that described by Noble et al. in U.S. Pat. No. 5,130,027, incorporated herein by reference, may be used. Alternatively, the magnetic particles may be contacted with the extract in a flowing stream, such as the outflow of a fermentor. Static or other mixing devices may be used to facilitate mixing.

The magnetic particles are then separated from the crude silk protein extract using a magnetic separator. In a magnetic separator, gradients in magnetic field strength are utilized to separate the magnetic particles from non-magnetic cell debris and other solids in the extract. The magnetic field gradient evokes a force on the magnetic particles such that the magnetic particles are deposited onto a surface, while the non-magnetic particles are not deposited, or such that the magnetic and non-magnetic particles are focused in different directions. Various types of magnetic separators are well known in the art (see for example, Svoboda, Magnetic Methods for the Treatment of Minerals, Elsevier, New York, 1987). Any suitable magnetic separator may be used in the method of the present invention, including but not limited to sidepull permanent magnets, rare earth magnets, electromagnetic separators, or high gradient magnetic separation (HGMS) separators, such as a Model L-1CN Frantz Canister Separator available from S. G. Frantz Co. (Trenton, N.J.). In one embodiment, a sidepull permanent magnet is used to separate the magnetic particles from a small extract volume, e.g., up to about 100 mL. In another embodiment, an HGMS separator is used to separate the magnetic particles from larger extract volumes. After magnetic separation of the magnetic particles, the extract is removed using any suitable method, including but not limited to decantation, filtering, or aspiration.

The magnetic particles are optionally washed with a suitable wash solution to reduce the cell debris content captured with the magnetic particles, thereby increasing the purity of the recovered silk protein. The wash step may be repeated one or more times. Any suitable buffer solution may be used for the wash, such as those described above for suspending the cell paste. Agents with high affinity to the magnetic particle surface that would compete with the binding of the silk protein, should not be present in the wash solution, or if present, their concentration should be low compared to their concentration in the elution solution. Specifically, agents that cause an alteration of the affinity ligand, causing a decrease in its binding affinity or selectivity towards the silk protein, should not be present in the wash solution, or if present, their concentration should be low compared to their concentration in the elution solution. For example, a chelating agent such as EDTA should not be present in the wash solution, or if present, its concentration should be low compared to its concentration in the elution solution, if a metal ion chelate is used as the affinity ligand. The particles are separated from the wash solution using magnetic separation as described above.

Then, an elution solution is added to the magnetic particles to recover the silk protein. The composition of the elution solution is such that the affinity of the affinity ligand for the affinity tag is significantly reduced, thereby releasing the bound silk protein from the magnetic particles. Any of the elution methods that are well known in the field of affinity chromatography may be used in the method of the present invention (see for example, Mohr et al. supra, Chapter 6). Specifically, the elution solution may contain an agent that has a high binding affinity for the affinity ligand which displaces the bound silk protein. Alternatively, the silk protein may be eluted using a pH change, a change in ionic strength, or by the addition of a chaotropic agent such as urea or guanidine. When a metal chelate affinity ligand is used, the elution solution may contain a competitive binding agent such as EDTA or imidazole. Alternatively, a low pH solution may be used for elution of the silk protein. In one embodiment, an elution solution containing imidazole is used to recover silk protein having a His tag from magnetic particles with immobilized IDA-Cu or NTA-Ni chelate. Optionally, the elution step may be repeated one or more times to increase the yield of silk protein.

The recovered silk protein may be used as is or may be further purified, concentrated, or dialyzed using methods known in the art, as needed for the intended use. If a cleavage site is included in the silk fusion protein, the affinity tag may be removed using enzymatic or chemical treatment, as is well known in the art.

Microbial Production with Excretion of Silk Protein

In another embodiment, the silk protein is excreted by the microbial host into the fermentation medium. For example, this is the case for silk proteins expressed in Bacillus subtilis, as described by Fahnestock (U.S. Pat. No. 6,268,169). In this embodiment, the magnetic particles are added directly to the fermentation medium without a cell disruption step and the purification process is performed as described supra. There is no need to remove the cells from the fermentation medium, although optionally the cells may be removed using methods known in the art, e.g., centrifugation or filtration.

Production of Silk Protein in Mammalian Cells

In another embodiment, the silk proteins are expressed in mammalian cells. The silk proteins may be expressed within the cells or secreted into the cell culture medium. In the former case, the cells are disrupted and the magnetic particles are added to the resulting crude silk protein extract, as described above for microbial expression. When the silk protein is excreted by the mammalian cells into the culture medium, the magnetic particles are added directly to the culture medium, as described above for microbial production with excretion into the fermentation medium.

Production of Silk Protein in Transgenic Animals

In another embodiment, the silk proteins are produced in transgenic animals. In this embodiment, the silk proteins are secreted in the milk or urine of the transgenic animal. The sample comprising the silk protein, including, but not limited to, milk or urine, is used directly in the purification method by adding the magnetic particles thereto and following the procedure described above.

Production of Silk Protein in Plants

In yet another embodiment, the silk proteins are expressed in plants. The silk proteins are purified and recovered from the plant tissue by first disrupting the plant tissue, preferably by physical means, including but not limited to, grinding or homogenization. The magnetic particles are then added to the resulting crude silk protein extract and the purification is performed as described above.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows “min” means minute(s), “h” means hour(s), “sec” means second(s), “psi” means pounds per square inch, “μL” means microliter(s), “mL” means milliliter(s), “L” means liter(s), “nm” means nanometer(s), “μm” means micrometer(s), “mM” means millimolar, “M” means molar, “mmol” means millimole(s), “μmole” means micromole(s), “g” means gram(s), “kg” means kilogram(s), “μg” means microgram(s) and “mg” means milligram(s), “g” means the gravitation constant, “vol %” means percent by volume, “A276” means the absorbance at a wavelength of 276 nm, “A260” means the absorbance at a wavelength of 260 nm, “rpm” means revolutions per minute, “kDa” means kilodaltons.

General Methods:

Materials and methods suitable for the maintenance and growth of bacterial cultures are well known in the art. Techniques suitable for use in the following Examples may be found as set out in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, DC (1994) or by Thomas D. Brock in Biotechnology: A Textbook of Industrial Microbiology, Second Edition, Sinauer Associates, Inc., Sunderland, Mass. (1989). All reagents and materials used for the growth and maintenance of bacterial cells were obtained from Aldrich Chemicals (Milwaukee, Wis.), BD Diagnostics Systems (Sparks, Md., formerly DIFCO Laboratories), Invitrogen Life Technologies (Carlsbad, Calif., formerly GIBCO/BRL), or Sigma Chemical Company (St. Louis, Mo.) unless otherwise specified.

All chemicals were reagent-grade and used as received from the manufacturer or distributor. Unless otherwise noted, the biochemicals were obtained from Sigma Chemical Company

### EXAMPLE 1

### Purification and Recovery of Spider Silk Analog Protein DP-2A Using Ferrite Magnetic Particles

The purpose of this Example was to demonstrate the recovery and purification of spider silk analog protein DP-2A using ferrite magnetic particles. The spider silk analog protein DP-2A was expressed in E. coli and was recovered and purified using magnetic affinity separation.

Production of Spider Silk Analog Protein DP-2A in E. coli:

E. coli strain FP3276 designed for the production of spider silk analog protein DP-2A (SEQ ID NO:4) as described by Fahnestock in U.S. Pat. No. 6,268,169, (incorporated herein by reference), was cultured as described in Example 5 of U.S. Pat. No. 6,268,169 (incorporated herein by reference) with minor modifications as follows. This spider silk protein analog was designed to contain six consecutive histidine residues (6×His tag) at the C-terminus for purification by metal chelate affinity techniques. Strain FP3276 was grown at 36° C. in a BioLafitte fermenter in 10 L of a medium as given in Table 1.

TABLE 1
Composition of Growth Medium Used for
Culturing Host E. coli Strains

| | | |
|---|---|---|
| (NH₄)₂SO₄ | 3.0 | g |
| MgSO₄ | 4.5 | g |
| Na citrate.2H₂O | 0.47 | g |
| FeSO₄.7H₂O | 0.25 | g |
| CaCl₂.2H₂O | 0.5 | g |
| Thiamine-HCl | 0.6 | g |
| Casamino acids | 200 | g |
| Biotin | 0.05 | g |
| K₂HPO₄ | 19.5 | g |
| NaH₂PO₄ | 9.0 | g |
| Glycerol | 100 | g |
| L-Alanine | 10.0 | g |
| Glycine | 10.0 | g |
| Glucose | 200 | g |
| Polypropylene glycol (Aldrich | 10 | mL |
| Chemical Co # 20,233-9) | | |
| ZnSO₄.7H₂O | 0.08 | g |
| CuSO₄.5H₂O | 0.03 | g |
| MnSO₄.H₂O | 0.025 | g |
| H₃BO₃ | 0.0015 | g |
| Ammonium molybdate.4H2O | 0.001 | g |
| CoCl₂.6H₂O | 0.0006 | g |

The fermenter was inoculated with 500 mL overnight culture of FP3276 in 2×YT (16 g Bacto-tryptone, 10 g Bacto-yeast extract, 5 g NaCl per liter at pH 7.0)+2% glucose+50 mg/L kanamycin. The pH was maintained at 6.8 by addition of 40% NH₄OH or 20% H₃PO₄. Dissolved O₂ was maintained at approximately 25%. When the absorption at 600 nm had reached 10-15, production of DP-2A was induced by adding 5 g IPTG (isopropyl β-D-thiogalactopyranoside) contained in 1 L of medium at 1/5 the concentration of original medium in the 10 L tank. After 3 h, the cells were harvested by centrifugation in a GS-3 type rotor in a Sorval Model RC5C refrigerated centrifuge (Kendro Laboratory Products, formerly Sorvall Instruments, Newtown, Conn.) and the cell paste was stored frozen at −20° C. for at least 24 h before proceeding with the purification process.

The cell paste (0.5 g) was thawed and resuspended in a total volume of 2.5 mL of cold phosphate buffer to give a concentration equivalent to that in the fermentation broth. Lysozyme was added to the cell suspension to a concentration of 1 mg/mL. DNase was added to a concentration of 1 U/mL and the solution was incubated at 8° C. for 30 min. Then, the cell suspension was sonicated on ice using a Vibracell sonicator (Sonic & Material, Danbury, Conn.) six times for 10 sec each with 5 sec pauses in between to give the crude silk protein extract.

Preparation of Ferrite Magnetic Particles:

Ferrite magnetic particles coated with polyglutaraldehyde were prepared using a method similar to that described by Hubbuch et al. (Biotechnol. Bioeng., 79(3):301-313 (2002)). An aqueous solution (S1) containing 0.25 mol/L iron(II) chloride and 0.5 mol/L iron(III) chloride (both obtained from Aldrich Chemical Co., Milwaukee, Wis.) was prepared. An aqueous solution (S2) containing 5 mol/L sodium hydroxide (Aldrich Chemical Co.) was prepared. Two hundred milliliters of each of solutions S1 and S2 were poured rapidly and simultaneously into a beaker containing 100 mL of deionized water, stirred by an overhead mixer. The mixing was provided by a stainless steel 2 bladed impeller rotating at 200 rpm driven by an overhead motor. A black precipitate formed instantly and after 5 min of mixing the iron oxide crystals were allowed to settle out of solution on a permanent magnet plate. The clear liquid above the magnetic particles was pumped off and discarded. The magnetic particles were washed repeatedly with large quantities of deionized water by cycles of resuspension, magnetic separation and decantation until the pH of the wash water dropped below 8.5. The iron oxide crystal slurry was subsequently washed once with a 0.2 mol/L sodium chloride solution and then twice with methanol before finally resuspending in 99 vol % methanol/1 vol % water to give a suspension with a crystal concentration of approximately 40 g/L.

The above crystal suspension was transferred to a glass beaker and homogenized for 5 min at 2000 rpm using a Polytron® PT6100 dispersing device (Kinematika AG, Littau, Switzerland) fitted with a Polytron® PT-DA 6030-6060 aggregate. Ten milliliters of 3-aminopropyltriethoxysilane (Aldrich Chemical Co, product no. 440140) was added, followed by the addition of 5 mL of glacial acetic acid. The homogenization speed was increased to 13,000 rpm for 10 min before reducing it to 6000 rpm for 2 h. The slurry was then mixed with 200 mL of glycerol (Aldrich Chemical Co, product no. 13,487-2) and stirred using an overhead mixer operated at 200 rpm for 5 min. The stirred content of the beaker was then heated to 110° C. on a standard laboratory hot plate under a nitrogen atmosphere in a fume hood over a period of approximately 90 min to evaporate the more volatile components. Once this temperature was reached, the mixture was cured for 10 h with gentle stirring under nitrogen. Finally, the temperature was raised to 160° C. over 30 min to evaporate excess silane. The mixture was then allowed to cool down to room temperature. The resulting amino-terminated magnetic particles were recovered from the glycerol suspending medium by 5 consecutive cycles of magnetic separation, decanting, and resuspension with deionized water.

The amino-terminated magnetic ferrite particles were coated with polyglutaraldehyde (PGA) in a manner similar to that described by Halling et al. (Biotechnol. Bioeng., 21:393-416 (1979)). In a fume hood, a 10 g portion of amino-terminated magnetic ferrite particles was stirred at room temperature with 1.75 L of an aqueous glutaraldehyde solution (2 vol %) for 1 h. The pH of the mixture was kept at 11 throughout the reaction by repeated addition of a 1 mol/L sodium hydroxide solution. The PGA-coated magnetic particles were recovered from the suspending medium by 6 consecutive cycles of magnetic separation, decanting, and resuspension with deionized water. The particle suspension was stored at 4° C. until required.

Iminodiactetic acid (IDA) was covalently bound to the PGA-coated magnetic particles by a three-step reaction scheme. These reaction steps consisted of an allyl-activation step, a bromination step and the IDA coupling step by nucleophilic substitution. A 2.2 g portion of PGA-coated particles was stirred in 120 mL of a mixture containing 50 vol % allyl glycidyl ether (AGE) (Aldrich Chemical Co.), 75 mmol/L sodium hydroxide (Aldrich Chemical Co.), 25 vol % dimethyl sulfoxide (Fluka Chemical Co., Milwaukee, Wis. ) and 11.4 mmol/L sodium borohydride (Aldrich Chemical Co.) for 48 h at room temperature. The allyl-terminated magnetic particles were recovered from the suspending medium by 10 consecutive cycles of magnetic separation, decanting, and resuspension with deionized water. Thereafter, the allyl-terminated particles were stirred in a 200 mL solution of N-bromosuccinimide (28 mmol/L) for 1 h. The particles were recovered from the suspending medium by 7 consecutive cycles of magnetic separation, decanting, and resuspension with deionized water. Thereafter, the particles (25 g/L) were stirred in a solution containing 1.75 mol/L IDA and 1 mol/L sodium carbonate for 72 h at room temperature. The particles were recovered from the suspending medium by 3 consecutive cycles of magnetic separation, decanting, and resuspension with deionized water. Thereafter, excess reactive groups were reacted with ethanolamine by suspending the particles in 1 mol/L ethanolamine aqueous solution at 4° C. for 48 h. The particles were recovered from the ethanolamine medium by 3 consecutive cycles of magnetic separation, decanting, and resuspension with deionized water. The finished particles were stored in a phosphate/salt buffer (20 mmol/L sodium phosphate, 1 mol/L sodium chloride, pH 6.8) at 4° C. until required.

Silk Protein Purification Procedure:

The magnetic IDA-terminated particles were charged with copper(II) ions before the silk protein purification. The magnetic particles were recovered from the storage buffer by magnetic separation, decanting and resuspension in acetate buffer (0.1 mol/L sodium acetate, 0.1 mol/L sodium chloride, pH 3.8) at a particle concentration of approximately 10 g/L. Thereafter, the particles were magnetically separated, decanted and resuspended and stirred for 30 min in a buffer containing 0.1 mol/L sodium acetate, 0.1 mol/L sodium chloride, 0.02 mol/L copper(II) chloride, pH 3.8. The particles were recovered by 3 cycles of magnetic separation, decanting and resuspension in deionized water. The particles were then magnetically separated, decanted and resuspended in a phosphate buffer (0.02 mol/L sodium phosphate, 0.1 mol/L sodium chloride, pH 6.8).

The magnetic particles (10 mg) were added to 1 mL of the crude silk protein extract containing 178 mg of the cell paste in phosphate buffer (50 mM NaH₂PO₄, 100 mM NaCl, 10 mM Imidazole, 0.05% Tween 20, pH 8.0). The mixture of magnetic particles and extract was gently mixed at 8° C. for 30 min. A vortex mixer (Vortex Genie 2, Scientific Industries, Ltd., Bohemia, N.Y.) was used for all mixing steps during protein purification. The particles were then separated magnetically as follows. A 12-tube magnetic rack (Qiagen Inc., Valencia, Calif.) was used for all magnetic separation steps together with 1.5 mL disposable plastic tubes. The supernatant was removed by aspiration. The particles were resuspended in wash buffer (50 mM NaH₂PO₄, 100 mM NaCl, 10 mM Imidazole, 0.05% Tween 20, pH 8.0) and were mixed for 2 min to wash the particles. The particles were separated from the wash buffer as described above and the wash process was repeated. The wash buffer from the wash steps was analyzed for protein content, as described below. The particles were then treated with 0.5 mL of the elution buffer, consisting of 50 mM NaH₂PO₄, 100 mM NaCl, 200 mM Imidazole, 0.05% Tween 20, pH 8.0, for 10 min with mixing. The particles were then separated magnetically as described above and the supernatant containing the purified spider silk protein was analyzed as described below. The elution step was repeated and the second elution supernatant was also analyzed as described below.

Analysis of Purified Spider Silk Analog Protein DP-2A:

The purity of the silk protein was determined using capillary electrophoresis using the Protein Plus 200 LabChip® protocol on the Model 2100 Bioanalyser (Agilent Technologies, Waldbronn, Germany). All protein samples were clarified by centrifugation for 10 min at 10,000×g prior to analysis using a Microspin 24S centrifuge (Kendro Laboratory Products, formerly Sorvall Instruments, Newtown, Conn.). The spider silk protein analog was detected after a retention time of 36 min, corresponding to an apparent protein size of 153 kDa. This reflects the size of the formed complex between DP-2A and SDS rather than the actual protein size of the DP-2A protein. Electropherogram images were created using the Agilent Bio Sizing software, version A.02.12 (S1292).

Results of Protein Analysis:

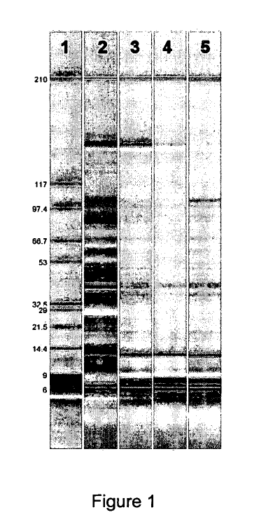

The Bioanalyser electropherogram of the protein composition of the crude silk protein extract and the purified fractions obtained using ferrite magnetic particles is shown in FIG. 1. Lane 1 represents a protein molecular weight standard. Lane 2 represents the E. coli cell extract before the addition of the magnetic ferrite particles. Lane 3 represents the supernatant after the first elution step. Lane 4 represents the supernatant after the second elution step. Lane 5 represents the supernatant of the first elution for a control experiment wherein the particles were not subjected to copper ions prior to the protein adsorption.

From lane 3, it can be clearly seen that the DP-2A protein is present in a concentrated and purified form compared to the crude silk protein extract shown in lane 2. The second elution step releases only minor amounts of additional DP-2A protein from the magnetic adsorbent particles as shown in lane 4. When the particles were not loaded with copper ions, no significant amount of DP-2A was bound and eluted from the magnetic particles as shown in lane 5. However, unspecific binding occurs to some degree using the PGA-coated ferrite particles.

### EXAMPLE 2

### Purification and Recovery of Spider Silk Analog Protein DP-2A Using Polyvinyl Alcohol-Coated Magnetic Particles

The purpose of this Example was to demonstrate the recovery and purification of spider silk analog protein DP-2A using polyvinyl alcohol (PVA)-coated magnetic particles. The spider silk analog protein DP-2A was expressed in E. coli and was recovered and purified using magnetic affinity separation.

Production of Spider Silk Analog Protein DP-2A in E. coli:

The spider silk analog protein DP-2A was prepared as described in Example 1.

Preparation of PVA-Coated Magnetic Particles:

Unfunctionalized magnetic polyvinyl alcohol (PVA) particles “M-PVA 01x” were purchased from Chemagen (Chemagen Biopolymer-Technologie AG, Baesweiler, Germany). Iminodiactetic acid (IDA) was covalently bound to the PGA-coated magnetic particles by a three-step reaction scheme. These reaction steps consisted of an allyl-activation step, a bromination step and the IDA coupling step by nucleophilic substitution. A 2.2 g portion of PGA-coated particles was stirred in 120 mL of a mixture of 50 vol % allyl glycidyl ether (AGE), 75 mmol/L sodium hydroxide, 25 vol % dimethyl sulfoxide and 11.4 mmol/L sodium borohydride for 48 h at room temperature. The allyl-terminated magnetic particles were recovered from the suspending medium by 10 consecutive cycles of magnetic separation, decanting, and resuspension with deionized water. Thereafter, the allyl-terminated particles were stirred in a 200 mL solution of N-bromosuccinimide (28 mmol/L) for 1 h. The particles were recovered from the suspending medium by 7 consecutive cycles of magnetic separation, decanting, and resuspension with deionized water. Thereafter, the particles (25 g/L) were stirred in a solution of 1.75 mol/L IDA and 1 mol/L sodium carbonate for 72 h at room temperature. The particles were recovered from the suspending medium by 3 consecutive cycles of magnetic separation, decanting, and resuspension with deionized water. Thereafter, excess reactive groups were reacted with ethanolamine by suspending the particles in 1 mol/L ethanolamine aqueous solution at 4° C. for 48 h. The particles were recovered from the ethanolamine medium by 3 consecutive cycles of magnetic separation, decanting, and resuspension with deionized water. The finished particles were stored in a phosphate/salt buffer (20 mmol/L sodium phosphate, 1 mol/L sodium chloride, pH 6.8) at 4° C. until required.

Purification and Analysis of Purified Spider Silk Analog Protein DP-2A:

The spider silk analog protein was purified and analyzed as described in Example 1.

Results of Protein Analysis:

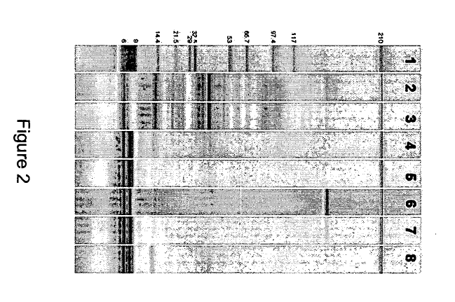

The Bioanalyser electropherogram of the crude silk protein extract and the purified fractions obtained using the magnetic M-PVA particles is shown in FIG. 2. Lane 1 represents a protein molecular weight standard. Lane 2 represents the E. coli cell extract before the addition of the magnetic M-PVA particles. Lane 3 represents the supernatant after the adsorption step. Lanes 4 and 5 represent the supernatant after the first and second washing step, respectively. Lanes 6 and 7 represent the supernatant after the first and second elution step, respectively. Lane 8 represents the supernatant of the first elution step for a control experiment wherein the particles were not subjected to copper ions prior to the protein adsorption.

From lane 3 it can be observed that the DP-2A protein has been selectively removed from the crude silk protein extract with the addition of the M-PVA particles when compared to the extract composition shown in lane 2.

The two washing steps of the magnetic particles to remove remaining cell debris did not cause a significant elution of DP-2A protein from the magnetic particles, as can be seen from lanes 4 and lane 5. From lane 6 it can be clearly seen that the DP-2A protein is present in a concentrated and highly purified form after subjecting the magnetic particles to the elution buffer. The second elution step released only minor amounts of additional DP-2A protein from the magnetic particles as shown in lane 7. When the particles were not loaded with copper ions, no significant amount of DP-2A protein was bound and eluted from the magnetic particles as shown in lane 8. Only a minor amount of an approximately 13 kDa protein was bound nonspecifically to the M-PVA particles when not loaded with copper ions prior to protein adsorption.

### EXAMPLE 3

### Investigation of the Effect of the Protein to Particle Ratio on the Purification of Spider Silk Protein Analog DP-2A

The purpose of this Example was to investigate the effect of the protein to particle ratio on the purification of spider silk analog protein DP-2A. The protein to particle ratio was varied from 4 to 32 mg of the spider silk analog protein per gram particle dry weight of the PVA-coated magnetic particles.

Spider silk analog protein DP-2A was produced as described in Example 1. The resulting crude silk protein extract was diluted to give a DP-2A protein concentration of 162 mg/L. The PVA-coated magnetic particles were prepared as described in Example 2. A series of purifications was carried out as described in Example 1 using protein to particle ratios of 4, 8, 16, and 32 mg of spider silk analog protein per gram dry weight of the PVA-coated magnetic particles. The DP-2A protein was eluted in a fifth of the volume (0.1 mL) compared to the volume in the adsorption step (0.5 mL).

The purified protein solutions were analyzed as described in Example 1.

Results of Protein Analysis:

The DP-2A protein concentrations and the total protein concentration were determined using the Agilent Bioanalyser. The purity of the DP-2A protein was calculated as the ratio of DP-2A protein mass in a sample to the mass of the total protein in the same sample. The average DP-2A purity was calculated as the arithmetic mean of the purities obtained in elution steps 1 and 2. The yield of the purification procedure was calculated as the ratio of DP-2A protein mass in the elution fraction to the DP-2A protein mass in the crude silk protein extract. The overall yield was calculated as the sum of the DP-2A protein masses in the two elution steps divided by the mass of DP-2A protein in the extract. Table 2 shows the purity and yield of the elution fractions with respect to the ratio of DP-2 protein in the extract and the M-PVA particle mass added to the extract in the adsorption step. High purities of 75-94% were achieved for all of the protein-to-particle ratios in the first elution steps. Similar purities of 72-91% were achieved in the second elution steps. The overall yields after two elution steps were in the range of 27-71%. The results show that as a general trend, the yield dropped with increasing ratio of protein to particles, whereas the purity increased at the same time. Therefore, optimization of the separation process may involve a compromise between purity and yield.

TABLE 2
Results of the Study of the Effect of the Protein to Particle
Ratio on the Purity and Yield of DP-2A Protein

| Ratio of DP-2A | DP-2A purity | DP-2A yield | DP-2A purity | DP-2A yield | Average | Overall |
|---|---|---|---|---|---|---|
| protein to | after first | after first | after second | after second | DP-2A | DP-2A |
| particle mass (mg/g) | elution step | elution step | elution step | elution step | purity | yield |
| 32 | 87% | 36% | 87% | 5% | 87% | 41% |
| 16 | 94% | 9% | 91% | 19% | 93% | 27% |
| 8 | 82% | 28% | 72% | 26% | 77% | 54% |
| 4 | 75% | 60% | 83% | 11% | 79% | 71% |

### EXAMPLE 4

### Comparative Example of the Purification and Recovery of Spider Silk Analog Protein DP-2A Using a Conventional Method

The purpose of this Example was to demonstrate the improved yield of spider silk analog protein DP-2A obtained with the magnetic affinity separation method of the instant invention compared to that obtained with a conventional purification method.

Conventional Purification Method

Two batches of cell paste (7.5 kg each), prepared as described in Example 1, were lysed by suspending each batch in 30 L of lysis buffer (50 mM Tris, 5 mM EDTA, pH 7.5). To each batch of slurry was added 750 mg of DNase and 0.225 g of magnesium chloride hydrate. Each batch was then processed in a Manton-Gaulin homogenizer, Model 15MR, at a feed rate of 0.3 L/min and an operating pressure of 5500 psi. The two batches of lysate were combined and given a second pass through the homogenizer, after which an additional 60 L of lysis buffer was added. Then, the diluted lysate was adjusted to pH 4.9 using acetic acid, heated to 65° C. and held at temperature for 15 min. The solids were separated from the protein-containing solution using membrane filtration with hollow-fiber membranes (Model CFP-2-E-65, A/G Technology, Needham, Mass.). The protein solution was then concentrated two steps using 10,000 nominal molecular weight cut-off, hollow-fiber membranes. Initial concentration was done using hollow-fiber membrane UFP-10-E-35 (UFP-10-E-35, Amersham Biosciences, Piscataway, N.J.). A flat-sheet membrane (Pellicon® 2 “Cassette” Filter, Type PCGC 10 K, Regenerated Cellulose, 0.5 m², Millipore Corp. Billerica, Mass.) was used in the second concentration step. Protein was precipitated using 1 volume of saturated ammonium sulfate to 9 volumes of protein solution at room temperature.

Analysis of Spider Silk Analog Protein DP-2A:

The protein concentration after each step in the purification process was determined using High Performance Liquid Chromatography (HPLC). The samples were diluted prior to analysis using either 20% acetonitrile, 0.2% trifluoroacetic acid in water, or 10% acetonitrile, 0.1% trifluoroacetic acid in water. In order to prevent the silk protein from precipitating, the trifluoroacetic acid concentration of the diluted sample should be about 0.1%. Therefore, samples that were diluted 1:1 were diluted with the 20% acetonitrile solution. Other dilutions, typically 1:3, 1:9, and 1:50, were made using either the 20% acetonitrile solution or the 10% acetonitrile solution. The analysis was done using an Agilent Model 1100 HPLC system (Agilent Technologies, Wilmington, Del.) with a Jupiter C4 column (5 μm particles, 30 nm pore size, 150×2 mm column, obtained from Phenomenex, Torrance, Calif.). The column was maintained at 35° C. The HPLC separation was achieved using a gradient combining two solvents: Solvent A, 0.2% trifluoroacetic acid in water and Solvent B, 0.2% trifluoroacetic acid in acetonitrile. The solvent gradient and the flow rates used are given in Table 3.

TABLE 3
Solvent Gradient and Flow Rates Used for HPLC Analysis

| | | | Flow Rate, |
|---|---|---|---|
| Time, min | Solvent A | Solvent B | mL/min |
| 0 | 80% | 20% | 0.3 |
| 8 | 20% | 80% | 0.3 |
| 9.5 | 80% | 20% | 0.5 |
| 14 | 80% | 20% | 0.3 |

Protein was detected at a wavelength of 230 nm. The silk protein eluted at 5.4 min in the assay. The silk protein concentration in ppm was obtained by dividing the peak area in the chromatogram by 8.16.
Results of Protein Analysis:

The results of the HPLC analysis were used to calculate the yield of silk protein after each step in the purification process. The resulting yields are summarized in Table 4.

TABLE 4
Yield of Spider Silk Analog Protein DP-2A Obtained
Using a Conventional Purification Method

| Processing Step | Step Yield % | Cumulative Yield % |
|---|---|---|
| Heating to 65° C. | 93.8 | 93.8 |
| Solid/Liquid Separation | 44.6 | 41.8 |
| Membrane | 85.5 | 35.8 |
| Concentration | | |
| Final Clean-up and | 91.1 | 32.6 |
| Concentration | | |
| Overall Yield % | | 32.6 |

These results demonstrate that the overall yield for the purification of spider silk analog protein DP-2A using the conventional method, i.e., 32.6%, is significantly lower than that obtained using the magnetic affinity method as described in Example 3, i.e., up to 71%. Moreover, the data shows that significant yield losses were observed for the steps that are eliminated using the magnetic affinity method, i.e., solid/liquid separation (loss of 55.4%) and concentration (loss of 23.4%).

1. A method for the purification of silk protein from a sample comprising:
a) providing a sample comprising at least one silk protein having an affinity tag in the presence of contaminating proteins;
b) contacting the sample with magnetic particles comprising an affinity ligand having a binding affinity for the affinity tag for a time sufficient for capture of the at least one silk protein onto the magnetic particles;
c) separating the magnetic particles from the sample by applying a magnetic field; and
d) recovering the silk protein from the magnetic particles by contacting the particles with an elution solution.

2. A method for the purification of silk protein from a host cell comprising:
a) providing a host cell comprising at least one silk protein having an affinity tag;
b) disrupting the host cell to release the at least one silk protein and produce a crude silk protein extract;
c) contacting the crude silk protein extract with magnetic particles comprising an affinity ligand having a binding affinity for the affinity tag for a time sufficient for capture of the silk protein onto the magnetic particles;
d) separating the magnetic particles from the crude silk protein extract by applying a magnetic field; and
e) recovering the silk protein from the magnetic particles by contacting the particles with an elution solution.

3. A method according to claim 1 or 2 wherein the magnetic particles are washed one or more times with a wash solution after step 1(c) or step 2(d).

4. A method according to claim 1 or 2 wherein the recovering step of 1(d) or 2(e) is repeated one or more times.

5. A method according to claim 1 or 2 wherein the affinity tag is selected from the group consisting of an antibody, an antibody fragment, glutathione S-transferase, a histidine tag, streptavidin, avidin, Protein A, Protein G, maltose-binding protein, a peptide having the amino acid sequence as set forth in SEQ ID NO:5, a peptide having the amino acid sequence as set forth in SEQ ID NO:6, and mixtures thereof.

6. A method according to claim 1 or 2 wherein the affinity ligand is selected from the group consisting of Protein A, Protein G, an antigen, IgG, an IgG fragment, glutathione, biotin, an antibody, an antibody fragment, amylose, a metal chelate, and mixtures thereof.

7. A method according to claim 6 wherein the metal chelate is a chelate of nitrilotriacetic acid or iminodiacetic acid with a heavy metal bivalent ion selected from the group consisting of Cu(II), Ni(II), Co(II), Zn(II), Hg(II), and Fe(II).

8. A method according to claim 1 or 2 wherein the affinity tag is a histidine tag and the affinity ligand is a metal chelate of iminodiacetic acid with Cu(II) or a metal chelate of nitrilotriacetic acid with Ni(II).

9. A method according to claim 1 or 2 wherein the magnetic particles are selected from the group consisting of magnetite, maghemitite, FePt, SrFe, iron, cobalt, nickel, chromium dioxide, ferrites, and mixtures thereof.

10. A method according to claim 1 or 2 wherein the magnetic particles are coated with a polymer or gel selected from the group consisting of polyethylene glycol, polymethacrylate, polymethylmethacrylate, polyethylenimine, polyvinyl alcohol, polyvinyl acetate, polystyrene, polyglutaraldehyde, polyacrylamide, agarose, chitosan, and alginate.

11. A method according to claim 1 or 2 wherein the magnetic particles have a diameter of about 2 nanometers to about 50 micrometers.

12. A method according to claim 1 or 2 wherein the magnetic particles have a diameter of about 100 nanometers to about 10 micrometers.

13. A method according to claim 1 or 2 wherein the magnetic field is applied by means selected from the group consisting of sidepull permanent magnets, rare earth magnets, electromagnetic separators, and high gradient magnetic separation separators.

14. A method according to claim 2 wherein the host cell is disrupted by mechanical means.

15. A method according to claim 14 wherein the mechanical means is selected from the group consisting of sonication, irradiation, homogenization, pressing, and freeze thawing.

16. A method according to claim 2 wherein the host cell is disrupted chemically.

17. A method according to claim 2 wherein the host cell is disrupted enzymatically.

18. A method according to claim 2 wherein the host cell is selected from the group consisting of prokaryotic cells, yeasts, fungi, algae, green plants, and mammalian cells.

19. A method according to claim 18 wherein the host cell is selected from the group consisting of Escherichia, Bacillus, Saccharomyces, Schizosaccharomyces, Pichia, Aspergillus, and Streptomyces.

20. A method according to claim 18 wherein the host cell is selected from the group consisting of soybean, rapeseed, pepper, sunflower, cotton, corn, tobacco, alfalfa, wheat, barley, oats, sorghum, rice, Arabidopsis, cruciferous vegetables, melons, carrots, celery, parsley, tomatoes, potatoes, strawberries, peanuts, grapes, grass seed crops, sugar beets, sugar cane, beans, peas, rye, flax, hardwood trees, softwood trees, and forage grasses.

21. A method according to claim 1 wherein the sample is selected from the group consisting of; milk, urine, and fermentation medium.

22. A method according to claim 1 or 2 wherein the silk protein is selected from the group consisting of the dragline spider silk proteins Spidroin 1 and Spidroin 2, spider silk proteins originating from the minor ampullate gland of Nephila clavipes, spider silk proteins originating from the flagelliform gland of Nephila clavipes and Argiope trifasciata, spider silk proteins originating from the major ampullate gland of Nephila madagascariensis, Nephila senegalensis, Tetragnatha kauaiensis, Tetragnatha versicolor, Argiope aurantia, Argiope trifasciata, Gasteracantha mammosa, Latrodectus geometricus, and Dolomedes tenebrosus, silk proteins originating from the silk glands of Plectreurys tristis and Euagrus chisoseus, and variants thereof.

23. A method according to claim 22 wherein the silk protein is a dragline spider silk protein.

24. A method according to claim 23 wherein the dragline spider silk protein is defined by the formula:
[AGQGGYGGLGXQGAGRGGLGGQGAGAnGG]z
wherein X=S, G or N; n=0-7 and z=1-75, and wherein the value of z determines the number of repeats in the variant protein and wherein the formula encompasses variations selected from the group consisting of:
(a) when n=0, the sequence encompassing AGRGGLGGQGAGAnGG is deleted;
(b) deletions other than the poly-alanine sequence, limited by the value of n will encompass integral multiples of three consecutive residues;
(c) the deletion of GYG in any repeat is accompanied by deletion of GRG in the same repeat; and
(d) where a first repeat where n=0 is deleted, the first repeat is preceded by a second repeat where n=6; and wherein the full-length protein is encoded by a gene or genes and wherein said gene or genes are not endogenous to the Nephila clavipes genome.

25. A method according to claim 23 wherein the dragline spider silk protein is defined by the formula: is defined by the formula:
[GPGGYGPGQQGPGGYGPGQQGPGGYGPGQQGPSGPGSAn]z
wherein n=6-10 and z=1-75 and wherein, excluding the poly-alanine sequence, individual repeats differ from the consensus repeat sequence by deletions of integral multiples of five consecutive residues consisting of one or both of the pentapeptide sequences GPGGY or GPGQQ and wherein the full-length protein is encoded by a gene or genes and wherein the gene or genes are not endogenous to the Nephila clavipes genome.

26. A method according to claim 24 wherein the dragline spider silk protein has a repeating unit having the amino acid sequence as set forth in SEQ ID NO:1, SEQ ID NO:2, or SEQ ID NO:3.

27. A method according to claim 25 wherein the dragline spider silk protein has a repeating unit having the amino acid sequence as set forth in SEQ ID NO:4.
